(19) United States
(12) Patent Application Publication
(10) Pub. No.: US 2025/0276444 A1
(43) Pub. Date: Sep. 4, 2025

(54) FORCE TRANSMISSION MECHANISMS AND RELATED DEVICES AND METHODS

(71) Applicant: INTUITIVE SURGICAL OPERATIONS, INC., Sunnyvale, CA (US)

(72) Inventor: William A. Burbank, Sandy Hook, CT (US)

(73) Assignee: INTUITIVE SURGICAL OPERATIONS, INC., Sunnyvale, CA (US)

(21) Appl. No.: 19/064,148

(22) Filed: Feb. 26, 2025

Related U.S. Application Data
(60) Provisional application No. 63/559,300, filed on Feb. 29, 2024.

(51) Int. Cl. B25J 9/10; B25J 9/12

(52) U.S. Cl. CPC B25J 9/109; B25J 9/126

(57) ABSTRACT

A steerable instrument includes a shaft comprising an articulable segment; a force transmission mechanism at the proximal end portion of the shaft, the force transmission mechanism comprising one or more rotatable drive components having axes of rotation spaced from the longitudinal axis of the shaft, the one or more rotatable drive components configured to be rotatably driven by respective drive input torques; one or more push-pull actuation elements configured to translate to transmit compressive forces to the articulable segment; and a rotatable coupler mechanism coupling one of the one or more rotatable drive components to the one or more push-pull actuation elements, the rotatable coupler mechanism configured to rotate about the longitudinal axis of the shaft in response to rotation of the one of the one or more rotatable drive components to cause translation of the one or more push-pull actuation elements.

### CROSS-REFERENCE TO RELATED APPLICATIONS

This application claims priority to U.S. Provisional Application No. 63/559,300, filed Feb. 29, 2024, which is incorporated herein by reference in its entirety.

### TECHNICAL FIELD

Aspects of the present disclosure relate to force transmission mechanisms and related devices and methods. For example, aspects of the present disclosure relate to force transmission mechanisms that convert rotational input forces to translational forces that articulate and/or actuate components of an instrument, such as a medical or other remotely actuatable instrument.

### INTRODUCTION

Various tools such as medical (including surgical) or industrial instruments often include shafts having one or more moveable structures along a length of the shaft or coupled to an end of the shaft. Such moveable structures can include joints or end effectors that impart one or more degrees of freedom of movement to such instruments. Such moveable structures are often actuated and controlled via translating actuation elements extending along a length of the shaft and coupled to the moveable structure at one end and to a force transmission mechanism at an opposite end. Such actuation elements may include, for example, cables or other tension members that operate in a pull-pull manner to transmit tensile (pulling) force acting on the member so as to actuate the moveable structure, or rods or other compression members that operate in a push-pull manner to transmit tensile (pulling) or compressive (pushing) force acting on the member so as to actuate the moveable structure.

Force transmission mechanisms are generally situated at a proximal end portion of the shaft of an instrument, with the end effector of the instrument situated at a distal end portion. Force transmission mechanisms can include a variety of driven force transmission components, such as capstans, gears, linkages, levers, and the like coupled to drive inputs that can be manually operated or configured to interface with a computer-assisted, teleoperated manipulator system.

In various applications, force transmission mechanisms include rotary input drive members (sometimes referred to as input drive disks). Rotation of rotary input drive members is then converted to translation of the actuation elements through various components and structures configured to transform rotational drive forces to translational forces to translate the actuation elements, thereby transmitting the desired force to articulate a moveable component of instrument.

Due to various architectural factors and packaging constraints, some instrument force transmission mechanisms have limited space. Moreover, some instrument force transmission mechanisms may have input drive members that are laterally offset from the instrument shaft and the actuation elements that are to be driven by the input drive members. In addition, the amount of force and force conversion needed in some of the limited space, relatively high friction environments found in some instruments pose challenges for robust and durable force transmission mechanisms.

There exists a need for force transmission mechanisms that are compact, mechanically robust, and able to be adapted to existing instrument shaft and manipulator designs. Additionally, there exists a need for such systems that possess desirable characteristics such as manufacturability and reliability in operation.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with at least one aspect of the present disclosure, A steerable instrument includes a shaft extending along a longitudinal axis from a proximal end portion to a distal end portion, the shaft comprising an articulable segment; a force transmission mechanism at the proximal end portion of the shaft, the force transmission mechanism comprising one or more rotatable drive components having axes of rotation spaced from the longitudinal axis of the shaft, the one or more rotatable drive components configured to be rotatably driven by respective drive input torques; one or more push-pull actuation elements coupled to the articulable segment and configured to translate to transmit compressive forces to the articulable segment to articulate the articulable segment; and a rotatable coupler mechanism coupling one of the one or more rotatable drive components to the one or more push-pull actuation elements, the rotatable coupler mechanism configured to rotate about the longitudinal axis of the shaft in response to rotation of the one of the one or more rotatable drive components to cause translation of the one or more push-pull actuation elements.

In another aspect of the present disclosure, a steerable instrument includes a shaft extending along a longitudinal axis from a proximal end portion to a distal end portion, the shaft comprising an articulable segment; a force transmission mechanism at the proximal end portion of the shaft, the force transmission mechanism comprising a rotatable drive component having an axis of rotation spaced from the longitudinal axis of the shaft, the rotatable drive component configured to be rotatably driven by a drive input torque; a pair of push-pull actuation elements coupled to the articulable segment and configured to translate so as to transmit compressive forces to the articulable segment to articulate the articulable segment; and a rotatable coupler mechanism coupling the rotatable drive component to the pair of push-pull actuation elements, the rotatable couple mechanism configured to rotate in response to rotation of the rotatable drive component to cause translation of the pair of push-pull elements in opposite directions.

In yet another aspect of the present disclosure, a steerable instrument includes a shaft extending along a longitudinal axis from a proximal end portion to a distal end portion, the shaft comprising an articulable segment; a first actuation element operably coupled to the articulable segment of the shaft and extending along the shaft from the articulable segment to the proximal end portion of the shaft; a second actuation element operably coupled to the articulable segment of the shaft and extending along the shaft from the articulable segment to the proximal end portion of the shaft; and a rotatable coupler mechanism rotatable about \the longitudinal axis of the shaft and comprising cam surfaces respectively engageable with the first and second actuation elements so as to drive the first and second actuation elements in translation in response to rotation of the rotatable coupler mechanism.

Yet another aspect of the present disclosure contemplates a method of articulating an articulable segment of an instrument shaft that includes driving rotation of a rotatable coupler mechanism via a rotatable drive component having an axis of rotation offset from an axis of rotation of the rotatable coupler mechanism, wherein the rotatable coupler mechanism is engaged with a push-pull actuation element coupled to the articulable segment; and in response to rotation of the rotatable coupler mechanism, driving translation of the push-pull actuation element so as to transmit a compressive force by the push-pull actuation element to cause articulation of the articulable segment.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 is a schematic, side view of an embodiment of an instrument comprising a force transmission mechanism.

FIG. 2 is a perspective view of an embodiment of a shaft of an instrument.

FIG. 3 is an enlarged view of a proximal end portion of the shaft of FIG. 2.

FIG. 4 is an interior view of a force transmission mechanism according to an embodiment.

FIG. 5A is a perspective view of an embodiment of a rotatable sleeve of the force transmission mechanism of FIG. 4.

FIG. 5B is a projection view of an interior surface of the rotatable sleeve of FIG. 5A.

FIG. 6 is a perspective view of a drive input of the force transmission mechanism of FIG. 4.

FIG. 7 is a perspective view of an embodiment of a crank arm of the force transmission mechanism of FIG. 4.

FIG. 8 is a perspective view of another embodiment of an instrument shaft.

FIG. 9 is a perspective, interior view of a force transmission mechanism according to another embodiment.

FIG. 10 is a perspective view of an embodiment of a manipulator system.

FIG. 11 is a partial schematic view of another embodiment of a manipulator system.

### DETAILED DESCRIPTION

Embodiments of the present disclosure relate to force transmission mechanisms that are configured to transmit drive inputs, received from computer-assisted (e.g., teleoperated) manipulators or manually-operated input devices, to forces to actuate actuation elements operably coupled to control movement of moveable components of an instrument, such as joints along an instrument shaft or an end effector. Force transmission systems according to various embodiments of the present disclosure enable drive input forces to occur at a location that is laterally offset from the longitudinal axis of the instrument shaft. Further, various embodiments of force transmission mechanisms can allow for transmission of force to multiple actuation elements in a relative limited and compact space. In addition, some embodiments of force transmission mechanisms according to the present disclosure include drive components that are configured to transmit drive forces from multiple drive inputs, each of which is configured to rotate about a respective axis, to actuation elements of a shaft arranged about a longitudinal axis of the shaft offset from the location of the axes of the drive inputs.

Embodiments of transmission systems according to the present disclosure may have particular relevance to shafts including non-cable type actuation elements, such as push-pull actuation elements. For example, some types of articulable shafts include concentric longitudinal tubular members that may be formed out of one or more layers of sheet metal. The articulable shafts may further include push-pull actuation elements formed or cut out of the one or more layers of sheet metal. Such push-pull actuation elements, unlike tension member (pull-pull type) actuation elements (such as cables or the like), generally cannot be routed around pulleys, capstans, etc. to easily change direction and route along a particular pathway dictated by the overall layout of the force transmission mechanism and instrument shaft. However, push-pull actuation elements may have certain advantages as compared to tension member (pull-pull type) actuation elements, such as but not limited to greater resistance to buckling and therefore the ability to transmit pushing, as well as pulling, force to a coupled component, and lower cost of manufacturing and/or assembly in an instrument. Various embodiments of transmission systems as disclosed herein may facilitate adaptation of shaft designs, including those incorporating layered sheets of push-pull type actuation elements to manipulator system designs typically used in conjunction with tension member (pull-pull) actuated instruments.

Force transmission systems of various embodiments may include mechanical drive component arrangements that transmit drive forces in the form of rotational forces about the axes of the drive inputs to a common axis of the instrument shaft about which the actuation elements are arranged. In some arrangements of instruments and associated manipulators, the axes of the drive inputs can be laterally offset from the common axis of the instrument shaft. In embodiments of the disclosure in which a single degree of freedom of movement of the shaft is actuated via two actuation elements moving in opposite directions (such as the use of a pair of opposing cables to effect pitch or yaw of a distal portion of the shaft), force transmission mechanisms of the present disclosure can provide simultaneous, opposing movement of each actuation element based on a single input, e.g., a rotational drive input. Embodiments of the disclosure provide such configurations under the space constraints imposed by some manipulator designs.

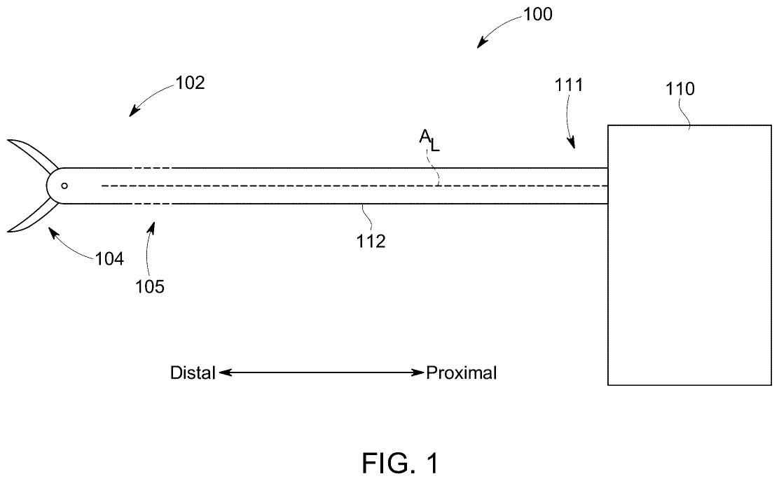

Referring now to FIG. 1, a schematic, side view of an elongate instrument 100 according to some embodiments is shown. Instrument 100 can be or include an instrument used to perform surgical, diagnostic, therapeutic, and other medical or non-medical procedures. The instrument 100 includes an end effector 104, a shaft 112 defining a longitudinal axis A_{L}, and a force transmission mechanism 110. The end effector 104 is located at a distal end portion 102 of the shaft 112. The end effector 104 can be configured to carry out a medical or non-medical (such as industrial) procedure. For example, the end effector 104 can include one or more tools such as gripping tools, staplers, shears, ligation clip appliers, electrosurgical tools, or other types of tools. While the illustration of FIG. 1 depicts an end effector having openable/closeable jaw members, such a configuration is exemplary and non-limiting and those of ordinary skill in the art would appreciate the instrument 100 can have any of a variety of end effectors without departing from the scope of the present disclosure

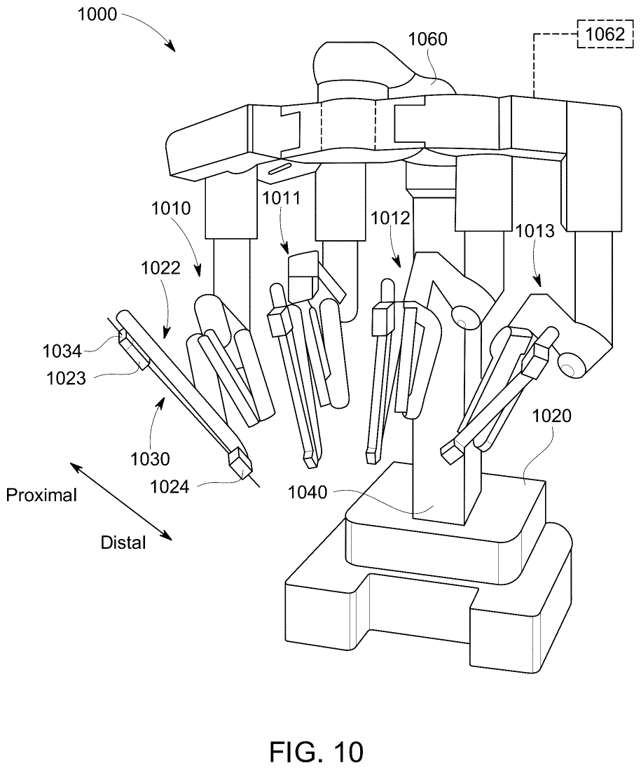

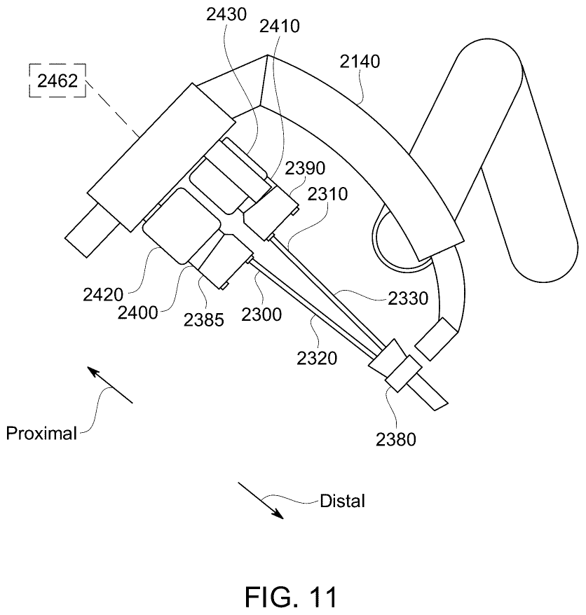

In the embodiment of FIG. 1, the force transmission mechanism 110 is coupled to a proximal end portion 111 of the shaft 112. In other embodiments, the force transmission mechanism 110 may be coupled at a mid-portion of the shaft 112, such as distal to a proximal end of the shaft 112. The force transmission mechanism 110 can be configured to be operably coupled with (e.g., removably coupled with) a computer-assisted (e.g., teleoperated) manipulator system, such as the manipulator systems described in further detail below in connection with FIGS. 10 and 11 or similar manipulator systems with which those having ordinary skill in the art are familiar. In other embodiments, in addition to or in lieu of being configured to interface and be driven by a computer-assisted manipulator system, the force transmission mechanism 110 can be manually controlled with manually operated (e.g., handheld) actuators (not shown).

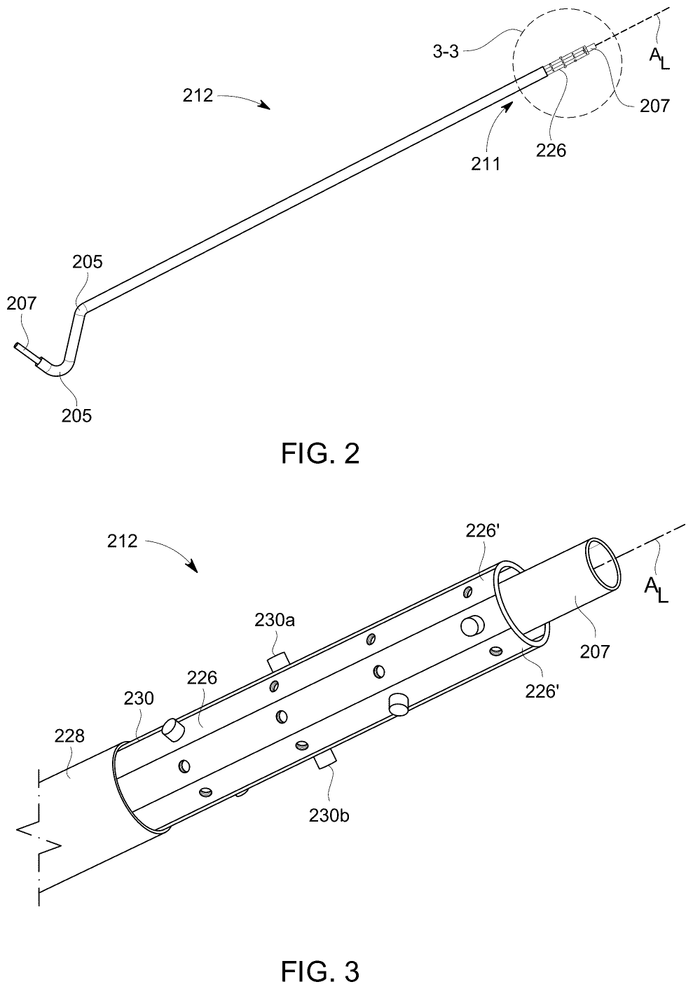

In the embodiment shown in FIG. 1, the instrument 100 includes an articulable segment 105 arranged along the shaft 112 between the end effector 104 and the force transmission mechanism 110. As shown in FIG. 1, the articulable segment 105 can be positioned toward the distal end portion 102 of the shaft 112. However, the disclosure is not so limited and the articulable segment 105 can be positioned at any location along the shaft 112 without limitation. In addition, the instrument 100 can include more than one articulable segment 105, such as two, three, or more articulable segments located at multiple locations along the length of the shaft 112. The articulable segment 105 can be controlled and actuated via actuation elements (not shown in FIG. 1; discussed above and in connection with the various embodiments of FIGS. 2-9) operably coupled to a manipulator by a force transmission mechanism. The articulable segment 105 can include one or more joints configured as flexible portions (e.g., sections of the shaft in which the material of the shaft is deflectable) of the shaft 112, or as structural joints (e.g., hinged or otherwise pivotable portions) of the shaft 112. The one or more articulable segments 105 can be articulated by actuation and control of the actuation elements to provide pitch and/or yaw motion to the end effector 104.

In some embodiments as discussed herein, the shaft 112, the one or more articulable segments 105, and actuation elements housed in the shaft 112 (not shown in FIG. 1; discussed in connection with the various embodiments of FIGS. 2-9) may have a configuration like that disclosed at least in, for example, U.S. Pat. No. 8,740,884 (issued Jun. 3, 2014) titled “INSTRUMENT FOR ENDOSCOPIC APPLICATIONS AND THE LIKE,” (the '884 patent); U.S. Pat. No. 8,986,317 (issued Mar. 24, 2015) titled “INSTRUMENT AND METHOD FOR MAKING THE SAME”; U.S. Patent App. Pub. No. 2012/0245414 (filed Mar. 23, 2011) titled “HANDLE FOR CONTROLLING INSTRUMENTS, ENDOSCOPIC INSTRUMENT COMPRISING SUCH A HANDLE, AND AN ASSEMBLY,” (the '414 publication), and U.S. Patent App. Pub. No. 2018/0008805 (filed Jun. 5, 2017) titled “METHOD FOR MANUFACTURING A STEERABLE INSTRUMENT AND SUCH A STEERABLE INSTRUMENT,” (the '805 publication), the entire contents of each of which are incorporated herein by reference. For example, the longitudinal elements 4 disclosed in the '805 application can correspond to actuation elements as discussed herein. Embodiments of the shafts disclosed in the patent and publications identified above can include push-pull type actuation elements formed of one or more layers of metal or other material, such as a laminated sheet metal actuation element, as discussed above. Moreover, other embodiments of shafts, such as those including tension member (pull-pull type) actuation elements and not related to the patent and publications identified above, are within the scope of the present disclosure.

Referring now to FIG. 2, an embodiment of a shaft 212 that can be used as shaft 112 is shown in isolation to better illustrate various aspects of the shaft 212. The shaft 212 extends generally along a longitudinal axis A_{L} and includes one or more articulable segments 205. As shown in FIG. 2, the articulable segments 205 can be located at a distal end portion 202 of the shaft 212. FIG. 2 is provided as exemplary in nature and in other embodiments, one or more articulable segments 205 can be positioned further proximally along the shaft, and any desired number and position of articulable segments is considered within the scope of the disclosure.

The distal end portion 202 of the shaft 212 can include an end effector (not shown) such as, but not limited to, tools such as grippers, staplers, shears, ligation clip appliers, electrosurgical tools, imaging tools (e.g., endoscopes), or other types of tools as described above with respect to the embodiment of FIG. 1. In some embodiments, the articulable segments 205 can provide pitch and/or yaw degrees of freedom, and the movement of the articulable segments 205 can be controlled to provide pitch and/or yaw motion to the end effector relative to the proximal portion of the shaft 212. The articulable segment 205 can be actuated to impart a desired geometry to the shaft 212 for accessing worksites, such as sites of interest in minimally invasive medical procedures. The shaft 212 can comprise multiple tubular elements or layers, including an inner layer 207, an intermediate layer 226, and an outer layer 228. The layers may have tubular configurations, such as hollow cylindrical configurations. The intermediate layer 226 is located radially between the inner layer 207 and the outer layer 228. The intermediate layer 226 includes push-pull actuation elements 226′ for controlling the instrument, such as those described above with respect to FIG. 1. In some embodiments, the intermediate layer itself may be further subdivided into multiple thinner layers having a laminate structure, which can facilitate the ability to bend with less bending stress and force, with each of the multiple thinner layers having one or more actuation elements 226′. The inner layer 207 and outer layer 228 provide structure and form to the shaft 212 and can comprise flexible segments throughout and/or only at the locations of the articulable segments 205, with rigid segments in other areas of the shaft 212. In some embodiments, reliefs in the layers can be used to affect rigidity.

An end effector (not shown in FIG. 2; e.g., end effector 104 shown in FIG. 1) can be coupled to the inner layer 207. The inner layer 207 is coupled to a force transmission mechanism (not shown), such as force transmission mechanism 110 of FIG. 1), and can be actuated to roll about the longitudinal axis A_{L} by the force transmission mechanism, and in turn impart a roll degree of freedom movement to the end effector about the longitudinal axis A_{L}. Inputs from a manipulator to which the force transmission mechanism is coupled can be provided to move one or more drive components of the force transmission mechanism to actuate the roll degree of freedom. In various embodiments disclosed herein, the inner layer 207 is rotationally decoupled from the intermediate layer 226 and the outer layer 228 such that rotation of the inner layer 207 about the longitudinal axis A_{L} does not cause a corresponding rotation of either the intermediate layer 226 (or the actuation elements 226′ thereof) or of the outer layer 228. Thus, the roll orientation of the inner layer 207 (and thus the orientation of the end effector) does not impact the overall configuration of the articulable segments 205 of the shaft 212. The inner layer 207 can have one or more central lumens (not shown) through which one or more actuation elements (not shown and other than actuation elements 226′), such as push-pull (e.g., compression members) and/or pull-pull (e.g., tension members) actuation elements, can be routed for actuating the end effector, as those having ordinary skill in the art are familiar with.

Control of the articulable segments 205 can be accomplished by application of translational forces to actuation elements within the shaft 212. As discussed above, the shaft 212 can include push-pull type actuation elements. In the embodiment of FIG. 2, actuation elements 226′ are exposed at the proximal end portion 211 of the shaft 212. FIG. 3 shows an enlarged view of the proximal end portion 211 of the shaft 212 and illustrates actuation elements 226′ arranged around the central longitudinal axis A_{L} of the shaft 212. The actuation elements 226′ can comprise, for example, actuation elements formed of a single layer or multiple laminated layers of sheet metal, as discussed in the '884 patent for example. Other types of actuation elements, such as pull-pull tension actuation elements (e.g., cables), push-pull compression member actuation elements (e.g., rods), and other elements can also be actuated using the force transmission mechanisms in accordance with various embodiments.

In various implementations, an articulable segment of a shaft can be articulated by actuation of a push-pull actuation element. In the embodiment of FIG. 2, each degree of freedom of the shaft 212 is associated with a pair of push-pull actuation elements 226′ located diametrically opposite one another relative to the axis A_{L}. Using such a pair of push-pull actuation elements 226′ can allow for one of the actuation elements 226′ to transmit a push force while the other transmits a pull force, thereby reducing the load on the main shaft. For example, a shaft including two articulable segments 205 (FIG. 2) each of which is separately actuatable in two degrees of freedom relative to the axis A_{L} (such as movement in pitch and yaw, respectively) may thus include eight actuation elements 226′ positioned around the shaft, as described further with reference to of the embodiment of FIG. 3. A pair of actuation elements 226′ generates movement in a single degree of freedom, such as one of pitch or yaw of one of the articulable segments 205, in response to opposing forces applied to the pair of actuation elements 226′. In other words, for each pair of actuation element 226 assigned to each degree of freedom, application of a distally-directed force (push force) to one actuation element 226 of the pair and a proximally-directed force (pull force) to the other actuation element 226 of the pair generates articulation of the articulable segment of the shaft in that degree of freedom. In some embodiments, movement of each degree of freedom is controlled by each pair of diametrically oppositely located actuation elements, but embodiments the present disclosure are not so limited. For example, the actuation elements could be spaced around the shaft 212 such that each degree of freedom is controlled by actuation elements spaced other than 180 degrees apart with respect to the shaft 212. Moreover, as noted above, any number of actuation elements can be used from 1 to more than 2 for actuation of an articulable segment and may be selected based on factors such as, but not limited to, overall load, space considerations, and complexity.

As discussed above, each actuation element 226′ is part of an intermediate layer 226 positioned between the outer layer 228 and the inner layer 207. Each actuation element 226′ is exposed from (extends beyond) the outer layer 228 at the proximal end portion 211 of the shaft 212 and extends distally to the articulable segment 205 with which the particular actuation element 226′ is associated. Each actuation element 226′ includes one or more features configured to interface with the force transmission mechanism to receive application of push or pull force to articulate the shaft 212 at the articulable segments 205.

For example, in FIG. 3, each of the actuation elements 226′ includes an engagement feature 230. As shown in FIG. 3, the engagement features 230 of different opposing pairs of actuation elements 226′ are staggered along the longitudinal axis A_{L} of the shaft 212. That is, with reference to a pair of engagement features 230a, 230b of an opposing pair of actuation elements 226′, the engagement features 230a, 230b are aligned along the axial direction (i.e., positioned diametrically opposite each other), but are spaced along the axial direction (not aligned) from engagement features 230 of the other opposing pairs of actuation elements 226′, and likewise for the engagement features 230 for each pair of opposing actuation elements 226′. Each engagement feature 230 is configured to engage with a component of the force transmission mechanism to receive actuation force from the transmission system, as discussed further below. As shown in FIG. 3, the engagement features 230 are in the form of protrusions, such as protruding pins configured to cooperate with a cam surface of the force transmission mechanism as is described further below.

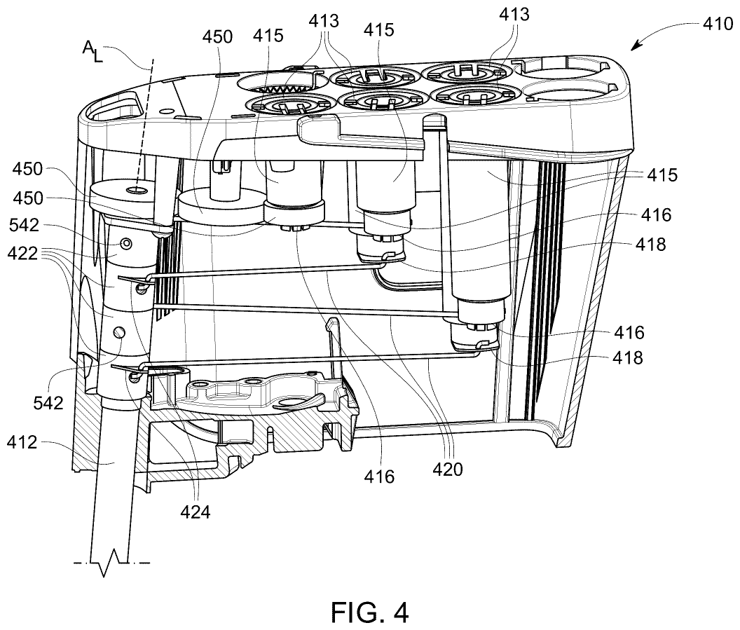

Referring now to FIG. 4, an embodiment of a force transmission mechanism 410 is shown. The force transmission mechanism 410 includes a chassis 414 to which the various components are coupled, and which is configured to couple to an instrument manipulator system (such as but not limited to manipulator systems as disclosed in connection with FIGS. 10 and 11 herein). In other embodiments (not illustrated), a force transmission mechanism can be configured with the internal drive components described for force transmission mechanism 410 but be modified to be driven via manual input mechanisms for use with manually operated instruments as those of ordinary skill in the art are familiar with. The force transmission mechanism 410 includes rotatable drive components in the form of drive input shafts 416 extending through shaft supports 415 of the chassis 414 and coupled to rotary drive disks at an exterior of the chassis 414. In an installed state of the force transmission mechanism 410 on a manipulator system, the input rotary drive discs, and thereby the drive input shafts 416, are configured to be operably coupled to drive outputs associated with the manipulator system, such as output drive discs or shafts. Rotation of the drive outputs of the manipulator system drives rotation of the input rotary drive disks and drive input shafts 416 of the force transmission mechanism 410. Also shown in FIG. 4 is a series of gears 450 (shown without teeth) that transmit torque from drive input shaft 416 to provide a roll degree of freedom to the inner layer 207 (FIG. 2) based on inputs from a manipulator system or manually, as discussed above.

As discussed above, the instrument shaft 412 is positioned along a central longitudinal axis A_{L} offset from the rotational axes of the respective drive input shafts 416. To impart motion to the instrument shaft 412 and end effector coupled at the distal end portion thereof (not shown in FIG. 4), motion of the drive input shafts 416 must be transferred from the drive input shafts 416 to actuation elements of the shaft 412. In the embodiment of FIG. 4, a mechanical linkage system is used to transfer the rotational motion of the drive input shafts 416 from rotational axes of the drive input shafts 416 to the actuation elements of the shaft 412, the longitudinal axis A_{L} of which is laterally spaced from the individual rotational axes of the drive input shafts 416.

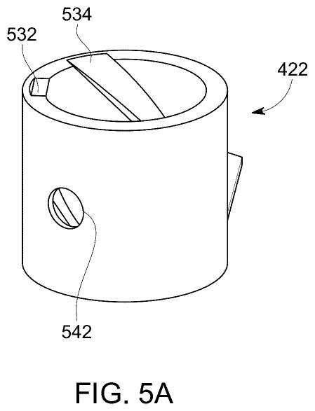

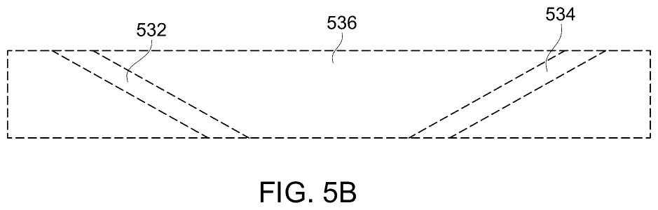

The mechanical linkages of the embodiment of FIG. 4 comprise a respective crank arm 418 (two of which are labeled in FIG. 4) to which each of the drive input shafts 416 is coupled. An embodiment of a crank arm 418 is shown in isolation in FIG. 7 and is described further below. Crank arm 418 is coupled to a first end of a connecting rod 420, with a second end of the connecting rod 420 being operably coupled to the instrument shaft 412 via a rotatable sleeve 422, shown in isolation in FIG. 5 and also described further below. The crank arm 418 is configured to be rotationally fixed with the drive input shaft 416 and to be held axially in position on the shaft 416.

The interface between the crank arm and the shaft can have various arrangements, such as, but not limited to splined interfaces, keyed interfaces, and any other suitable hub-shaft type interface so as to provide a fixed rotational relationship between the shaft 416 and the crank arm 418. Further, in some embodiments, the crank arms 418 could be made integral with the drive input shafts 416, or provided in any manner familiar to those having skill in the art.

The mechanical linkage components of the force transmission mechanism 410 further include one or more rotatable coupler mechanism that are coupled to a pair of actuation elements 226′ (FIGS. 2 and 3) such that rotation of a rotatable coupler mechanism causes translation of a pair of actuation elements 226′ in opposite directions to each other. For example, referring again to FIG. 4, a plurality of rotatable coupler mechanisms are provided in the form of rotatable sleeves 422 arranged in series around the shaft 412 along the longitudinal axis A_{L}. Each of the rotatable sleeves 422 cooperates with a different pair of opposing actuation elements 226′, and the axial spacing of the engagement features 230 of the actuation elements is arranged to achieve respective cooperation with the rotatable sleeves 422, as explained further below. Each of the rotatable sleeves 422 includes a flange 424 extending radially from an exterior of the rotatable sleeve 422. Finally, the mechanical linkage of the force transmission mechanism 410 further comprises a connecting rod 420 coupling a respective flange 424 and respective crank arm 418. Thus, each drive input shaft 416 is operably coupled to a respective pair of opposing actuation elements (such as actuation elements 226′, not shown in FIG. 4) of the shaft 412.

In various embodiments, a rotatable sleeve 422 may be coupled to fewer or more than two actuation elements 226′. For example, a single rotatable sleeve may be coupled to one, two, three or more actuation elements 226′. Further, different rotatable sleeves may be coupled to a different number of actuation elements 226′. For example, a first rotatable sleeve 416a may be coupled to two actuation elements 226′, while a second rotatable sleeve 416b may be coupled to one actuation element 226′. The numbers and arrangements depicted in the various illustrations herein are exemplary and non-limiting.

Each of the rotatable sleeves 422 can include features configured to interact with engagement features 230 (FIG. 3) of the actuation elements 226′ (FIG. 3). For example, each of the rotatable sleeves 422 can be configured to drive an opposing pair of actuation elements 226′ to articulate the shaft 412 in a degree of freedom with which the opposing pair of actuation elements 226′ is associated.

Reference is now made to FIG. 5A, which shows a perspective view of a rotatable sleeve 422, and FIG. 5B, which shows a projection of the interior surface of the rotatable sleeve 422 onto the plane of FIG. 5B (i.e., the interior surface of the rotatable sleeve 422 “unrolled” onto the plane of the drawing). Each of the rotatable sleeves 422 includes a first cam surface feature 532 and a second cam surface feature 534. Each of the first and second cam surface features 532 and 534 can be configured as a recessed slot on an interior wall (e.g., bore) 536 of the rotatable sleeve 422 arranged and dimensioned to receive and engage an individual engagement feature 230 (FIG. 3) of the actuation element 226′. More specifically, the cam surface features 532 and 534 can be configured to respectively receive the engagement features 230 (FIG. 3) of the pair of opposing actuation elements 226′ with which the sleeve 422 cooperates. The cam surface features 532 and 534 are in the form of inclined recesses angled, for example at least partially spiraling (following a helical path), relative to the longitudinal axis in opposite directions. That is, when viewed along the longitudinal axis AL of the shaft 212, the cam surface features 532 and 534 rotate in opposite directions about the longitudinal axis AL of the shaft 212 as the sleeve 422 rotates.

The pitch of the first and second cam surface features 532 and 534 can be chosen based on the overall desired translational movement of the actuation elements 226′, the range of motion of the drive input shafts 416, the force required to move the actuation elements 226′, and any requirement that the actuation elements 226′ be back-drivable (i.e., whether an external force applied to the shaft 212 can articulate the shaft 212), and other factors. In some exemplary embodiments, the first and second cam surface features 532 and 534 follow a helical path. In alternate embodiments, a rotatable sleeve 422 may have fewer or more than two cam surface features. For example, a rotatable sleeve 422 may have a single cam surface feature to couple to a single actuation element 226′, or a rotatable sleeve 422 may have three or more cam surface features to enable coupling to three or more respective actuation elements 226′. The angle of the helical pitch of the cam surface features relative to the axis of the sleeve can be selected based on various instrument specifications, such as torque, space constraints, and other design criteria those having ordinary skill in the art would be familiar with. Differing pitches can affect the speed at which the actuation element coupled thereto moves and cam surface features within a sleeve can have the same or different pitches, and likewise with cam surface features of different sleeves.

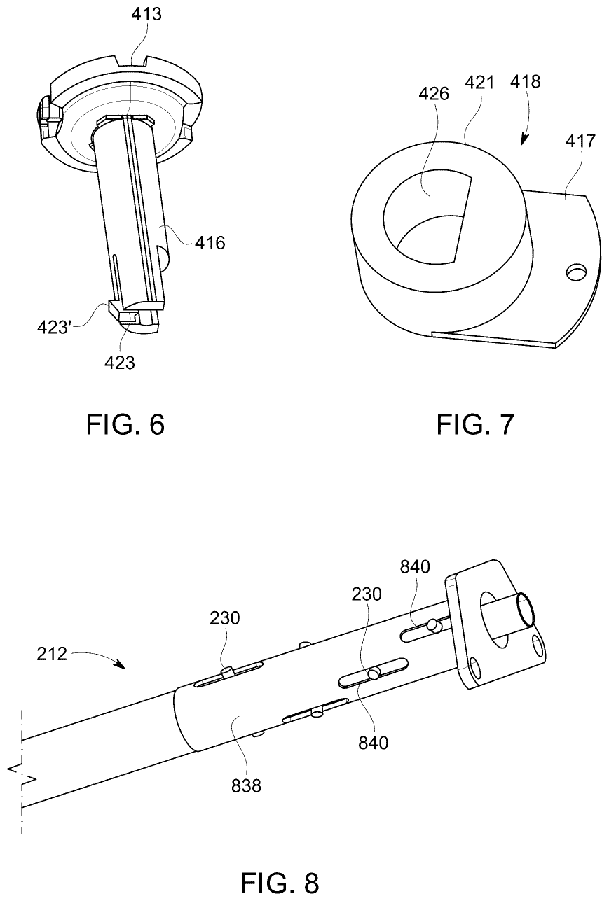

FIGS. 6 and 7 show isolated views of the drive input shaft 416 and crank arm 418. The drive input shaft 416 (FIG. 6) includes a drive input disc 413 configured to engage a drive output of a manipulator system as discussed above. Alternatively, the drive input shaft 416 could be configured to be driven by a manual input. The shaft 416 includes the resilient, radially inwardly deflectable clip 423 at the free end portion of the shaft 416. The shaft 416 has a cut out cross-section at the distal end portion thereof which is configured to mate with a complementary cross-section of a bore 426 of a hub portion 421 of the crank arm 418. For example, the bore 426 and the shaft 416 can each have a flat surface portion that interface with each other to provide an anti-rotational interface (e.g., D-shaped cross sections) between the two components. Upon insertion of the shaft 416 within the bore 426 of the crank arm 418, the surface of the bore 426 engages a protrusion 423′ on the resilient clip 423 which has an angled surface allowing the hub portion 421 of the crank arm 418 to slide up the distal end portion of the shaft 416, deflecting the clip 423. Once the hub portion 421 passes the protrusion 423, the resilient clip 423 deflects back in place and the protrusion 423 provides a stop surface preventing the crank arm 418 from sliding axially off the distal end portion of the shaft 416 and thereby retaining the crank arm 418 on the shaft 419, as shown in FIG. 4.

The cross-sectional configurations of the bore 426 of the crank arm 418 and the distal portion of the shaft 416 provides an interface that orients the shaft 416 and crank arm 418 relative to each other and prevents relative rotation between the shaft 416 and the crank arm 418. The configuration of the drive input shaft 416 and crank arm 418 described above may contribute to low part count and ease of assembly of the force transmission mechanism 410. As noted above, the particular configurations of the bore and shaft cross-sections are not required and various other arrangement for coupling the drive input shaft and crank arm in a rotationally fixed relationship is within the scope of the disclosure, such as a splined interface, keyed interface, a pin interface such as roll pin, or other arrangements as would be familiar to those having ordinary skill in the art. Further, while the drive input shaft 416 and crank arm 418 are shown and described as separate components, in other embodiments, the drive input shaft 416 and crank arm 418 can optionally be formed integrally or in other configurations as would be apparent to those having ordinary skill in the art. Further, as described above, additional or alternative connections may be used in conjunction with or alternative to the resilient clip 423, such as such as fasteners, adhesive, a press fit, or other connection.

In use, rotation of a drive input shaft 416 results in concurrent rotation of a rotatable sleeve 422 with which the drive input shaft 416 is associated via a respective connecting rod 420. Rotation of the rotatable sleeve 422 causes the cam surface features 532, 534 to bear against the respective engagement features 230 of the actuation elements 226′, thereby causing translational movement of the pair of actuation elements 226′ with which the rotatable sleeve 422 cooperates. For example, rotation of the rotatable sleeve 422 causes a first one of the pair of actuation elements 226′ to translate in a first direction (e.g., proximally), and a second one of the pair of actuation elements 226′ to translate in a second direction opposite to the first direction (e.g., distally) based on the direction of the helical recesses and the rotational direction of the drive input shaft 416, resulting in articulation of the shaft 212 in the degree of freedom with which the pair of actuation elements 226′ is associated (e.g., pitch or yaw). Articulation of the shaft 412 in the same degree of freedom, but in the opposite direction, can be achieved by reversing the direction of rotation of the drive input shaft 416 associated with that degree of freedom so that rotation of the rotatable sleeve 422 and translational movement of the corresponding opposing pair of actuation elements 226′ is reversed.

As will be appreciated by one of ordinary skill in the art, rotation of the rotatable sleeves 422 imparts loads on the engagement features 230 not only in the direction of desired movement of the actuation elements 226′, i.e., along the longitudinal axis A_{L} of the shaft 212, but also in lateral directions perpendicular to the longitudinal axis A_{L} of the shaft 212. The force transmission mechanism 410 (FIG. 4) can include additional structural features configured to support the actuation elements 226′ and lessen deflection of the actuation elements 226′ resulting from such side-loading. For example, referring now to FIG. 8, the shaft 212 may be provided with a tubular support member 838. The tubular support member 838 is configured to surround the actuation elements 226′ (FIG. 3) in the region of the engagement features 230 and includes slots 840 elongated in the axial direction of the shaft 212 and through which the engagement features 230 pass. In the assembled state shown in FIG. 4, the rotatable sleeves 422 surround the tubular support member 838, and the engagement features 230 protrude through the slots 840 and into the cam surface features 532 and 534 of each rotatable sleeve 422. The slots 840 constrain movement of the engagement features 230 to move longitudinally (parallel to the axial direction A_{L}) while supporting the engagement features 230 and actuation elements 226′ from side loads resulting from rotation of the rotatable sleeves 422.

Due to the presence of the tubular support member 838 and the opposite helical directions of the cam surface features 532 and 534, the engagement features 230 have constrained motion within the assembly. Various components of the force transmission mechanism 410 can include features configured to facilitate assembly of the components. For example, each of the rotatable sleeves 422 (FIG. 5A) can include one or more assembly ports 542 through which the engagement features 230 may be pushed and pass into the longitudinal slots 840 of the tubular support member 838 and then into one of a series of holes provided in the actuation elements 226′. The engagement features 230 can be separate from the actuation elements 226′ and pressed into one of the series of holes in the actuation elements with a press fit, an interference fit, or other means of connection to the actuation elements 226′. In other embodiments, the engagement features 230 may be made integrally with the actuation elements. Rotation of the sleeves 422 can then occur until the engagement feature is positioned in a corresponding cam surface feature 532, 534.

In the embodiment of FIGS. 4 and 5, the rotatable sleeves 422 are rotatable members configured with a common axis of rotation generally coaxial with the longitudinal axis A_{L} of the shaft 212. In other embodiments, actuator assemblies of the force transmission mechanism can include rotatable members that do not share a common axis of rotation and do not rotate about axes parallel to or coaxial with the longitudinal axis of the shaft 212.

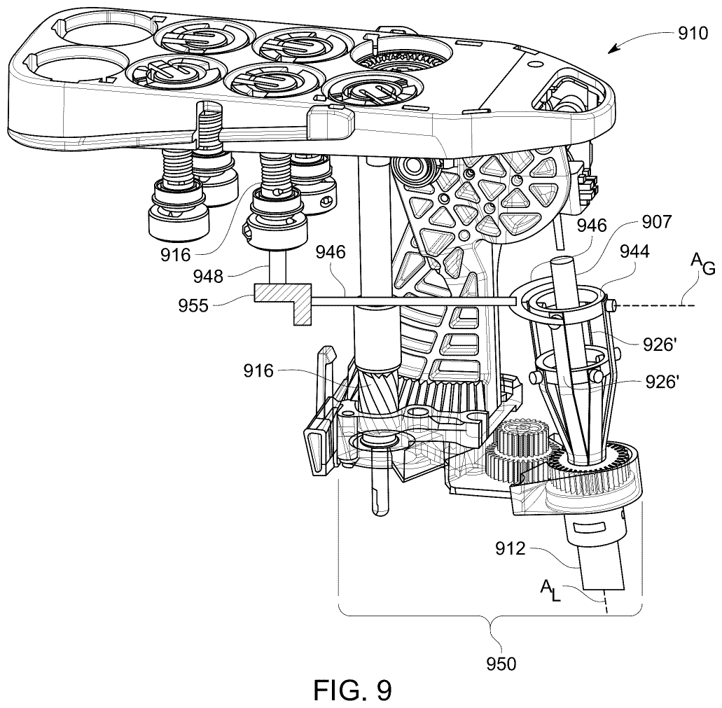

For example, with reference now to FIG. 9, another embodiment of a force transmission mechanism 910 according to the present disclosure is shown. In this embodiment, a rotatable coupler mechanism comprising a gimbal is utilized to transmit rotational movement of the rotary drive input to the translational motion of push-pull actuation elements. More specifically, a pair of actuation elements 926 that work together in tandem to create the opposite pushing/pulling forces can be coupled to differing locations of a rotatable gimbal 944. Similar to the embodiment of FIGS. 4-8, the embodiment of FIG. 9 can include eight actuation elements 926 to actuate four independent degrees of freedom of the shaft 912 via translational movement of each of a pair of actuation elements 926 working in a coordinated fashion in response to rotation of the gimbal. But, as discussed above, any number of actuation elements can be utilized to articulate a degree of freedom, including a single actuation element, to more than two for any particular degree of freedom.

In this embodiment, translation of a pair of actuation elements 926′ is accomplished by rotating the gimbal 944 about an axis intersecting the longitudinal axis of the shaft A_{L} and perpendicular to a line extending through the actuation elements 926′ desired to be actuated. For example, in the embodiment of FIG. 9, if labeled actuation elements 926′ are desired to be actuated, the gimbal 944 is rotated about axis A_{G}, thereby causing longitudinal movement of the actuation elements 926 in opposite directions. In the embodiment of FIG. 9, the gimbal 944 can be rotatable about two perpendicular axes defined by the locations at which the actuation elements 926′ are attached to the respective gimbal 944. Similar to the arrangement of the rotatable sleeves in the above embodiments, plural gimbals 944 could be used and at differing locations long the longitudinal axis of the shaft to engage with and transmit force to different ones or sets of actuation elements. Depending on the location of the actuation elements about the longitudinal axis A_{L}, articulation of an articulable segment can occur in differing directions and about differing axes (degrees of freedom).

The gimbal 944 is rotated in the desired manner via yoke 946, with. each gimbal 944 being rotated by two rotatable yokes 946 (only one yoke 946 shown in FIG. 9) to actuate the two degrees of freedom associated with each gimbal 944. Each rotatable yoke 946 is coupled to a driveshaft 948, which is in turn operatively coupled to a drive input 916 by a bevel gear set 955. Rotation of the drive input 916 drives rotation of the driveshaft 948 and yoke 946, thereby generating longitudinal movement of the actuation elements 926 in opposite directions to articulate the shaft 212 (FIG. 2). In some embodiments, multiple driveshafts 948 can be provided in a nested coaxial configuration depending on the number of degrees of freedom of the shaft to be articulated. As with other embodiments described above, the shaft 912 of FIG. 9 can include an inner layer 907 configured to impart roll motion to the shaft 912. More specifically, the inner layer 907 can be coupled to a drive input 916 through a series of gears 950 (e.g., planetary and stacked gear train in the embodiment of FIG. 9) and rotary input can be transmitted from the roll drive input 916 through the gear train 950 to impart roll motion to the inner layer 907 and thereby the shaft 912.

Force transmission systems according to embodiments of the present disclosure provide compact and relatively simple systems that can be adapted to various existing manipulator and shaft architecture. Features of force transmission systems according to the present disclosure can facilitate manufacturing, assembly, and contribute to generally low part count and relatively high reliability. Further, embodiments of force transmission mechanisms discussed herein can be used with actuation elements other than actuation elements 226′, 926′ discussed herein. For example, embodiments of force transmission mechanisms as discussed herein can optionally be used with cable-type actuation elements, such as pull-pull type actuation elements, compression-type actuation elements (e.g., rod elements), or other types of actuation elements.

Embodiments described herein may be used, for example, with remotely operated, computer-assisted systems (such, for example, teleoperated surgical systems) such as those described in, for example, U.S. Pat. No. 9,358,074 (filed May 31, 2013) to Schena et al., entitled “Multi-Port Surgical Robotic System Architecture”; and U.S. Pat. No. 9,295,524 (filed May 31, 2013) to Schena et al., entitled “Redundant Axis and Degree of Freedom for Hardware-Constrained Remote Center Robotic Manipulator”, each of which is hereby incorporated by reference in its entirety. Further, embodiments described herein may be used, for example, with any of the da Vinci® Surgical System commercialized by Intuitive Surgical, Inc., of Sunnyvale, California.

The embodiments described herein are not limited to the surgical systems noted above, and various other teleoperated, computer-assisted surgical system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As discussed above, in accordance with various embodiments, transmission systems of the present disclosure are configured for use in teleoperated, computer-assisted surgical systems employing robotic technology (sometimes referred to as robotic surgical systems). Referring now to FIG. 10, an embodiment of a manipulator system 1000 of a computer-assisted surgical system, to which surgical instruments are configured to be mounted for use, is shown. Such a surgical system may further include a user control system, such as a surgeon console (not shown) for receiving input from a user to control instruments coupled to the manipulator system 1000, as well as an auxiliary system, such as auxiliary systems associated with the da Vinci® surgical systems noted above.

As shown in the embodiment of FIG. 10, a manipulator system 1000 includes a base 1020, a main column 1040, and a main boom 1060 connected to main column 1040. Manipulator system 1000 also includes a plurality of manipulator arms 1010, 1011, 1012, 1013, which are each connected to main boom 1060. Manipulator arms 1010, 1011, 1012, 1013 each include an instrument mount portion 1022 to which an instrument 1030 may be mounted, which is illustrated as being attached to manipulator arm 1010. While the manipulator system 1000 of FIG. 10 is shown and described having a main boom 1060 to which the plurality of manipulator arms are coupled and supported thereby, in other embodiments, the plurality of manipulator arms can be coupled and supported by other structures, such as an operating table, a ceiling, wall, or floor of an operating room, etc.

Instrument mount portion 1022 comprises a drive assembly 1023 and a cannula mount 1024, with a transmission mechanism 1034 (which may generally correspond to the force transmission mechanisms 110, 410, 910 discussed in connection with FIGS. 1, 4, and 9) of the instrument 1030 connected to the drive assembly 1023, according to an embodiment. Cannula mount 1024 is configured to hold a cannula 1036 through which a shaft 1032 of instrument 1030 may extend to a surgery site during a surgical procedure. Drive assembly 1023 contains a variety of drive and other mechanisms that are controlled to respond to input commands at the surgeon console and transmit forces to the transmission mechanism 1034 to actuate the instrument 1030. Although the embodiment of FIG. 10 shows an instrument 1030 attached to only manipulator arm 1010 for ease of viewing, an instrument may be attached to any and each of manipulator arms 1010, 1011, 1012, 1013.

Other configurations of surgical systems, such as surgical systems configured for single-port surgery, are also contemplated. For example, with reference now to FIG. 11, a portion of an embodiment of a manipulator arm 2140 of a manipulator system with two surgical instruments 2300, 2310 in an installed position is shown. The surgical instruments 2300, 2310 can generally correspond to instruments discussed above, such as instrument 100 disclosed in connection with FIG. 1. The schematic illustration of FIG. 11 depicts only two surgical instruments for simplicity, but more than two surgical instruments may be mounted in an installed position at a manipulator system as those having ordinary skill in the art are familiar with. Each surgical instrument 2300, 2310 includes a shaft 2320, 2330 that at a distal end has a moveable end effector or an endoscope, camera, or other sensing device, and may or may not include a wrist mechanism (not shown) to control the movement of the distal end.

In the embodiment of FIG. 11, the distal end portions of the surgical instruments 2300, 2310 are received through a single port structure 2380 to be introduced into the patient. As shown, the port structure includes a cannula and an instrument entry guide inserted into the cannula. Individual instruments are inserted into the entry guide to reach a surgical site.

Other configurations of manipulator systems that can be used in conjunction with the present disclosure can use several individual manipulator arms. In addition, individual manipulator arms may include a single instrument or a plurality of instruments. Further, as discussed above, an instrument may be a surgical instrument with an end effector or may be a camera instrument or other sensing instrument utilized during a surgical procedure to provide information, (e.g., visualization, electrophysiological activity, pressure, fluid flow, and/or other sensed data) of a remote surgical site.

Force transmission mechanisms 2385, 2390 (which may generally correspond to force transmission mechanism 110 disclosed in connection with FIG. 1, force transmission mechanism 410 disclosed in connection with FIG. 4, and force transmission mechanism 910 disclosed in connection with FIG. 9) are disposed at a proximal end of each shaft 2320, 2330 and connect through a sterile adaptor 2400, 2410 with drive assemblies 2420, 2430. Drive assemblies 2420, 2430 contain a variety of internal mechanisms (not shown) that are controlled by a controller (e.g., at a control cart of a surgical system) to respond to input commands at a surgeon side console of a surgical system to transmit forces to the force transmission mechanisms 2385, 2390 to actuate surgical instruments 2309, 2310.

The embodiments described herein are not limited to the embodiments of FIG. 10 and FIG. 11, and various other teleoperated, computer-assisted surgical system configurations may be used with the embodiments described herein. The diameter or diameters of an instrument shaft and end effector are generally selected according to the size of the cannula with which the instrument will be used and depending on the surgical procedures being performed.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A steerable instrument, comprising:
a shaft extending along a longitudinal axis from a proximal end portion to a distal end portion, the shaft comprising an articulable segment;
a force transmission mechanism at the proximal end portion of the shaft, the force transmission mechanism comprising one or more rotatable drive components having axes of rotation spaced from the longitudinal axis of the shaft, the one or more rotatable drive components configured to be rotatably driven by respective drive input torques;
one or more push-pull actuation elements coupled to the articulable segment and configured to translate to transmit compressive forces to the articulable segment to articulate the articulable segment; and
a rotatable coupler mechanism coupling one of the one or more rotatable drive components to the one or more push-pull actuation elements, the rotatable coupler mechanism configured to rotate about the longitudinal axis of the shaft in response to rotation of the one of the one or more rotatable drive components to cause translation of the one or more push-pull actuation elements.

2. The steerable instrument of claim 1, wherein the rotatable coupler mechanism comprises a cam surface that drives the one or more push-pull actuation elements to translate the one or more push-pull actuation elements.

3. The steerable instrument of claim 2, wherein rotatable coupler mechanism comprises an inclined recess on an interior wall of the rotatable coupler mechanism.

4. The steerable instrument of claim 2, further comprising:
a plurality of push-pull actuation elements; and
a plurality of cam surfaces configured to drive the plurality of push-pull actuation elements,
wherein the plurality of cam surfaces are configured to drive at least some of the plurality of push-pull actuation elements at differing rates from each other.

5. The steerable instrument of claim 1, wherein the rotatable coupler mechanism comprises:
a first rotatable sleeve comprising a first plurality of cam surfaces engageable with a first pair of push-pull actuation elements and configured to drive translation of the first pair push-pull actuation elements in opposite directions in response to rotation of the first rotatable sleeve, and
a second rotatable sleeve comprising a second plurality of cam surfaces engageable with a second pair of push-pull actuation elements to drive translation of the second pair of push-pull actuation elements in opposite directions in response to rotation of the second rotatable sleeve, the second rotatable sleeve coupled to another of the one or more rotatable drive components.

6. The steerable instrument of claim 5, wherein the first rotatable sleeve and the second rotatable sleeve are each coaxial with the longitudinal axis of the shaft, the first rotatable sleeve being distal to the second rotatable sleeve.

7. The steerable instrument of claim 5,
wherein the first and second plurality of cam surfaces comprise respective first and second inclined recesses on an interior wall of the first rotatable sleeve and the second rotatable sleeve, respectively,
wherein each of the first pair of push-pull actuation elements comprises a protrusion configured to be received within the first inclined recess, and
wherein each of the second pair of push-pull actuation elements comprises a protrusion configured to be received within the second inclined recess.

8. The steerable instrument of claim 1, wherein the rotatable coupler mechanism comprises a gimbal which is coupled to a plurality of the push-pull actuation elements.

9. The steerable instrument of claim 8, wherein:
the one or more push-pull actuation elements comprises a first pair of push-pull actuation elements;
the steerable instrument comprises a second pair of push-pull actuation elements;
the first pair of push-pull actuation elements is coupled to the gimbal at a first pair of locations on the gimbal such that rotation of the gimbal about a first axis actuates the first pair of push-pull actuation elements; and
the second pair of push-pull actuation elements is coupled to the gimbal at a second pair of locations on the gimbal such that rotation of the gimbal about another axis actuates the second pair of push-pull actuation elements.

10. A steerable instrument, comprising:
a shaft extending along a longitudinal axis from a proximal end portion to a distal end portion, the shaft comprising an articulable segment;
a first actuation element operably coupled to the articulable segment of the shaft and extending along the shaft from the articulable segment to the proximal end portion of the shaft;
a second actuation element operably coupled to the articulable segment of the shaft and extending along the shaft from the articulable segment to the proximal end portion of the shaft; and
a rotatable coupler mechanism rotatable about the longitudinal axis of the shaft and comprising cam surfaces respectively engageable with the first and second actuation elements so as to drive the first and second actuation elements in translation in response to rotation of the rotatable coupler mechanism.

11. The steerable instrument of claim 10, wherein the articulable segment is articulatable in response to translation of the first actuation element and the second actuation element in opposite directions to each other.

12. The steerable instrument of claim 10, further comprising a third actuation element and a fourth actuation element, the third actuation element and the fourth actuation element coupled to the articulable segment of the shaft.

13. The steerable instrument of claim 12, wherein:
the rotatable coupler mechanism comprises a first rotatable sleeve comprising first cam surfaces engageable with the first and second actuation elements and a second rotatable sleeve comprising second cam surfaces engageable with the third and fourth actuation elements.

14. The steerable instrument of claim 13, wherein:
the articulable segment is articulable in a first degree of freedom based on translation of the first actuation element and the second actuation element in opposite directions to each other; and
the articulable segment is articulable in a second degree of freedom, differing from the first degree of freedom, based on translation of the third actuation element and the fourth actuation element in opposite directions to each other.

15. The steerable instrument of claim 10, further comprising a rotatable drive component having an axis of rotation offset from the longitudinal axis of the shaft, wherein the rotatable coupler mechanism is operably coupled to be driven in rotation in response to rotation of the rotatable drive component.

16. The steerable instrument of claim 15, wherein the rotatable coupler mechanism comprises a rotatable sleeve comprising the cam surfaces and operably coupled to the rotatable drive component.

17. The steerable instrument of claim 15, wherein the rotatable drive component is a first rotatable drive component and the axis of rotation is a first axis of rotation, and wherein the steerable instrument further comprises a second rotatable drive component having a second axis of rotation offset from the first axis of rotation and the longitudinal axis.

18. The steerable instrument of claim 17, wherein the rotatable coupler mechanism comprises:
a first rotatable sleeve operably coupled to be driven in rotation by the first rotatable drive component and comprising a cam surface engageable with the first actuation element, and
a second rotatable sleeve operably coupled to be driven in rotation by the second rotatable drive component and comprising another cam surface engageable with the second actuation element.

19. The steerable instrument of claim 18, wherein:
a first cam surface of the rotatable coupler mechanism is operably coupled to the first actuation element and is configured to be driven in rotation by the first rotatable drive component, and
a second cam surface of the rotatable coupler mechanism is operably coupled to second actuation element and is configured to be driven in rotation by the second rotatable drive component.

20. The steerable instrument of claim 15, wherein the cam surfaces comprise a first recess and a second recess engageable with respective first and second protrusions of the first and second actuation elements.
